(19) United States
(12) Patent Application Publication
(10) Pub. No.: US 2015/0056634 A1
(43) Pub. Date: Feb. 26, 2015

(54) Methods for Preparing Dry Formulations of Glucose Binding Protein

(71) Applicant: Becton, Dickinson and Company, Franklin Lakes, NJ (US)

(72) Inventors: Matthew S. Ferriter, Chapel Hill, NC (US); Javier Alarcon, Durham, NC (US); Glenn P. Vonk, Fuquay-Varina, NC (US); Melody M.H. Kuroda, Durham, NC (US); Mark Foster Sistare, Apex, NC (US); Brandi Marie Ford, Apex, NC (US)

(73) Assignee: Becton, Dickinson and Company, Franklin Lakes, NJ (US)

(21) Appl. No.: 14/388,957

(22) PCT Filed: Mar. 28, 2013
(86) PCT No.: PCT/US2013/034302

§ 371 Date: Sep. 29, 2014

Related U.S. Application Data
(60) Provisional application No. 61/616,737, filed on Mar. 28, 2012.

(51) Int. Cl. G01N 33/66; C07K 14/245

(52) U.S. Cl. CPC G01N 33/66; C07K 14/245
USPC 435/7.9; 530/350; 436/501; 435/7.1

(57) ABSTRACT

Methods and compositions for preparing dry formulations of Glucose Binding Proteins (GBPs) are disclosed. The GBPs may be stored as a dry formulation without significant loss of activity. After reconstitution, the GBPs may be used to determine the glucose concentration of a sample.

### BACKGROUND

Protein stability is an important consideration when determining protocols for preparation, purification and storage of proteins. Proteins are fragile molecules that often require great care during handling and storage to ensure that they remain intact and fully active. The secondary and tertiary structures of a protein, which represent the folding of the protein onto itself as a result of the nature of the side chains of the amino acids that form the protein, can be disrupted during routine handling of the protein. Critical parameters that are usually considered when handling or storing a protein include the components and pH of the protein buffer, the temperature of the environment in which the protein is handled, forces exerted on the protein (such as shaking and stirring), the total concentration of proteins in the solution, proteases in the environment, and the like.

More particularly, native glucose binding proteins (GBPs) derived from E. coli undergo significant degradation near or above their protein melting temperature (Tm), typically in the range of 35° to 50° C. in solution, which reduces the accuracy of GBP-based glucose assays. Storage and shipping conditions of glucose assays can approach about 50° C., which causes nearly complete loss of GBP activity in solution.

Although a number of proteins have been stabilized in dry formulations by lyophilization, there are no reports of GBP being stabilized in this way. One major reason for this is that the GBP must retain its conformational flexibility after being immobilized or trapped in a dry formulation. Also, carbohydrate excipients are closely related to glucose in structure and are expected to attenuate glucose response on reconstitution of the GBP.

### SUMMARY

The presently disclosed subject matter describes methods for preparing dry formulations of glucose binding proteins. The presently disclosed formulations do not require glycerol for the stability of the GBPs. After being dried, the GBP formulations can be used in glucose assays to determine the amount of glucose in a sample without the need for any additional reagents.

In one aspect, the presently disclosed subject matter describes a method for preparing a dry formulation of a GBP, the method comprising drying a solution comprising a GBP and wherein the GBP is folded in an active conformation. The solution comprising the GBP may or may not comprise a carbohydrate excipient.

In another aspect, the presently disclosed subject matter provides a method for determining an amount of glucose in a liquid sample, the method comprising drying a solution comprising at least one GBP, wherein the GBP is folded in an active conformation, reconstituting the GBP with the liquid sample, and measuring the amount of glucose in the liquid sample.

In a further aspect, the presently disclosed subject matter provides a dry formulation of GBP made by a process comprising drying a solution comprising at least one GBP, wherein the GBP is folded in an active conformation. The GBP may then be reconstituted with a liquid.

Certain aspects of the presently disclosed subject matter having been stated herein above, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

Having thus described the presently disclosed subject matter in general terms, reference will now be made to the accompanying Figures, which are not necessarily drawn to scale, and wherein:

FIG. 1 is a bar graph showing the stability of a GBP 3M-NBD with the carbohydrate excipient trehalose after lyophilization (Lyo), quench freezing in liquid nitrogen and then lyophilization (QF Lyo), and spray freeze drying and then lyophilization (SFD). GBP activity was determined after 0, 2, 4, 8, 12, or 24 weeks at room temperature and at 34° C.;

FIG. 2 is a graph showing the stability of a hydrogel-GBP W183C Acrylodan-trehalose dry formulation after 12 weeks of storage. The formulation was allowed to polymerize, disks were excised from the polymerized formulation, dried by lyophilization, and stored dessicated in a low water vapor transmission rate (WVTR) package at room temperature. After 12 weeks, the disks were placed in microwell plates and assayed for GBP activity in PBS containing various concentrations of glucose;

FIG. 3 is a graph showing the stability of a GBP 3M-NBD-trehalose formulation in microwell plates after 24 weeks of storage. The formulation was dispensed in microwells, dried under vacuum and stored dessicated at room temperature. After 24 weeks, the formulation was assayed for GBP activity in PBS containing various concentrations of glucose;

FIG. 4 is a graph showing the use of dry GBP formulations in a rapid glucose assay in 100% human plasma with no reagent additions. Microwell plates were prepared with a dried GBP W183 Acrylodan-trehalose formulation, serum was added to the microwells, and fluorescence evaluated; and

FIG. 5 is a bar graph showing the stability of GBP 3M-NBD with or without the carbohydrate excipient sorbitol in a hydrogel after lyophilization. GBP activity was determined in microwells after 0, 1, 3, 7, 14, 21, and 28 days at 25° C., 37° C., and 55° C.

### DETAILED DESCRIPTION

The presently disclosed subject matter now will be described more fully hereinafter with reference to the accompanying Figures, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

Typically, protein reagents are handled at room temperature or below, and stored at low temperatures (−20° C. to −70° C.) in the presence of aqueous glycerol. Protein stability may be assessed using activity assays or circular dichroism to detect temperature induced protein denaturation. Temperature stability is routinely described using the protein melting temperature, Tm, which is the temperature at which half of the protein is denatured.

Lyophilization (freeze drying) is widely used for long term protein storage. In this process, the solvent is removed by sublimation and the concentrated protein is reduced to a dehydrated powder, which can then be stored in a cold environment. This process needs to occur rapidly to avoid protein denaturation caused by concentration gradients of the components of the solvent, substantial shifts of pH, protein degradation at the protein-ice interface, and the like.

Excipients, which are pharmacologically inactive substances used as carriers in a formulation, can be used in the solvent during lyophilization (Lai and Topp, 1999). Representative excipients include carbohydrate excipients, such as trehalose (e.g. U.S. Pat. No. 4,891,319), sorbitol, mannose, and the like, and other kinds of excipients, such as proteins and synthetic polymers. Typically, carbohydrate excipients, such as trehalose, which are used to enhance proteins in dry formulations, are well tolerated in humans (Richards et al., 2002). A number of mechanisms have been proposed to explain the propensity of carbohydrate excipients to enhance temperature stability of proteins in dry formulations. In general, carbohydrates with high glass transition temperatures (T_{G}) are most useful in these formulations. One explanation for the correlation of high T_{G} and enhanced thermal stability is that the protein is immobilized in a glass that damps molecular motion as temperature increases, thereby preventing protein denaturation.

Glucose monitoring is important for individuals, such as diabetics, who require frequent monitoring of their glucose levels. Currently, commercialized glucose test strips are used for electrochemical determination of blood glucose, which involves detecting the oxidation of blood glucose using an enzyme, such as glucose oxidase. In general, glucose test strips for use in electrochemical based glucose monitors involve working and reference electrodes formed on the surface of the substrate, and a means for making a connection between the electrodes and the meter. The working electrode is coated with an enzyme capable of oxidizing glucose and a mediator compound that transfers electrons from the enzyme to the electrode resulting in a measurable current when glucose is present. Formulations include paste or ink, which is applied to the substrate. In the case of disposable glucose strips, this application is done by screen printing to obtain the thin layers suitable for a small flat test strip. Unlike a thicker carbon paste electrode, which remains fairly intact during the measurement, screen printed electrodes are prone to break up on contact with the sample. As a result, the components of the electrode formulation are released into solution and, once these components drift more than a diffusion length away from the underlying conductive layer, they no longer contribute to the measurement.

Although commercialized glucose strips are capable of a rapid response in less than five seconds, electrochemical methods are limited by the need to attain reproducible glucose diffusion profiles before glucose levels may be estimated. Often, materials used to control glucose diffusion in electrochemical assays incorporate additional functions: to limit oxygen diffusion, reduce interference from electrochemically active endogenous materials, and to ensure that the current is passed efficiently from the detector enzyme to the working electrode. As described above, electrochemical assays also are complicated by polarization dynamics at the electrode surface. Current glucose oxidase-based sensors have glucose sensitivities usually in the millimolar range.

Another protein that can detect glucose molecules is Glucose Binding Protein (GBP). The GBP from Escherichia coli (E. coli) is a periplasmic protein with a molecular weight of about 32 kDa (Stepanenko et al., 2011). It has a monomeric structure that folds in two main domains linked by three strands commonly referred to as the “hinge region” and a sugar-binding region that is located in the cleft between the two domains. This protein can bind both glucose and galactose with micromolar affinity, although the sensitivity of GBP to galactose is at a lower affinity than for glucose. The use of GBP in a glucose assay also has the advantage of reducing interferences from endogenous materials.

Generally, GBP activity depends on conformational changes of the GBP protein. In the absence of glucose, GBP exists in a number of conformers that converge toward a single conformer when glucose occupies its GBP binding site. Conformational changes involving the hinge are thought to be necessary for sugars to enter and/or exit the sugar-binding region.

The relative populations of GBP and GBP/glucose conformers may be detected using an environmentally sensitive fluorescent reporter dye placed at a defined position on a GBP. The dye changes fluorescence behavior (intensity and/or emission wavelength) according to the conformational status of GBP in the presence or absence of glucose. Therefore, GBP is useful for in vitro and in vivo glucose tests.

### I. METHODS OF PREPARING DRY FORMULATIONS OF GBP AND METHODS OF THEIR USE IN DETERMINING GLUCOSE CONCENTRATIONS

A. Methods of Preparing Dry Formulations of GBP

The detection of glucose in a biological sample can be used to diagnose and manage diabetes, where increased glucose monitoring can improve the long-term prognosis of subjects suffering from diabetes, including Type 1 and Type 2 diabetes. As described herein, current glucose monitoring methods use glucose oxidase and test strips that are assayed in electrochemical meters. The presently disclosed subject matter provides a method for preparing a dry formulation of GBP, such that glucose levels can be assayed in a biological sample after reconstitution of the GBP without the need for other reagents.

Up to now, it has been generally believed that GBPs could not be stored in a dry formulation and retain activity upon reconstitution because GBP exists in a number of conformations that converge toward a single conformer when glucose occupies the binding site of GBP. Therefore, GBP must retain its conformational flexibility after being immobilized or trapped in a dry formulation.

In addition, the carbohydrate excipients that are generally used for stabilizing proteins in dry formulations have molecular structures that are similar to the structure of glucose. Therefore, it is expected that after reconstitution of GBP, a carbohydrate excipient would interfere with the ability of GBP to detect glucose.

The presently disclosed subject matter provides methods for preparing dry formulations of GBP that can then be used after reconstitution to detect glucose molecules. It was found that GBP can be dried and stored for long periods of time and still be folded in an active conformation, as seen by the retention of significant activity, or lack of a significant loss of activity, after the GBP was reconstituted and assayed. In some embodiments, a loss of significant activity means more than a 10% loss of activity, more than a 20% loss of activity, or more than a 30% loss of activity as compared to the GBP in the original solution before the solution is dried. In a particular embodiment, it is meant that the GBP does not lose more than 20% activity as compared to the GBP in the original solution. GBP can be dried with or without excipients and without the presence of glycerol as a stabilizing agent.

The methods relate to GBPs in general, such as the GBP from E. coli, which also is called the D-galactose/D-glucose-binding protein (GGBP). A wide variety of GBPs can be used in the methods. For example, thermophilic GBPs, such as tmGBP from Thermotoga maritima, can be dried and stored for periods of time without loss of significant activity. It is expected that thermophilic GBPs are even more stable at higher temperatures than the E. coli GBP using the methods of the presently disclosed subject matter.

The GBP used in the methods may be a wild-type protein (meaning that no amino acids have been changed in the sequence as compared to the protein isolated from its original organism) or it may be a mutant protein that has one or more amino acids that have been changed. The term “mutant protein” is used herein as it is in the art. In general, a mutant protein can be created by addition, deletion or substitution of the wild-type primary structure of the protein or polypeptide. Mutations include for example, the addition or substitution of cysteine groups, non-naturally occurring amino acids, and replacement of substantially non-reactive amino acids with reactive amino acids. The amino acid residues that are changed may be found at the glucose binding site of GBP or in another part of the protein. Substitutions not at the glucose binding site of the GBP may still change the phenotype of the protein and/or allow a marker to be incorporated into the GBP. More than one amino acid substitution may be made in one GBP. The substitution may replace one or more amino acids naturally found in the GBP for an amino acid, such as cysteine, which is not found naturally in the GBP.

Embodiments include mutations of the E. coli GBP protein having a cysteine substituted for lysine at position 11 (K11C), a cysteine substituted for aspartic acid at position 14 (D14C), a cysteine substituted for valine at position 19 (V19C), a cysteine substituted for asparagine at position 43 (N43C), a cysteine substituted for glycine at position 74 (G74C), a cysteine substituted for tyrosine at position 107 (Y107C), a cysteine substituted for threonine at position 110 (T110C), a cysteine substituted for serine at position 112 (S 112C), a double mutant including a cysteine substituted for serine at position 112 and serine substituted for leucine at position 238 (S112C/L238S), a cysteine substituted for lysine at position 113 (K113C), a cysteine substituted for lysine at position 137 (K137C), a cysteine substituted for glutamic acid at position 149 (E149C), a double mutant including a cysteine substituted for glutamic acid at position 149 and an arginine substituted for alanine at position 213 (E149C/A213R), a double mutant including a cysteine substituted for glutamic acid at position 149 and a serine substituted for leucine at position 238 (E149C/L238S), a double mutant including a serine substituted for alanine at position 213 and a cysteine substituted for histidine at position 152 (H152C/A213S), a cysteine substituted for methionine at position 182 (M 182C), a cysteine substituted for alanine at position 213 (A213C), a double mutant including a cysteine substituted for alanine at position 213 and a cysteine substituted for leucine at position 238 (A213C/L238C), a cysteine substituted for methionine at position 216 (M216C), a cysteine substituted for aspartic acid at position 236 (D236C), a cysteine substituted for leucine at position 238 (L238C) a cysteine substituted for aspartic acid at position 287 (D287C), a cysteine substituted for arginine at position 292 (R292C), a cysteine substituted for valine at position 296 (V296C), a triple mutant including a cysteine substituted for glutamic acid at position 149 and a serine substituted for alanine at position 213 and a serine substituted for leucine at position 238 (E149C/A213S/L238S), a triple mutant including a cysteine substituted for glutamic acid at position 149 and an arginine substituted for alanine at position 213 and a serine substituted for leucine at position 238 (E149C/A213R/L238S), a cysteine substituted for glutamic acid at position 149 and a cysteine substituted for alanine at position 213 and a serine substituted for leucine at position 238 (E149C/A213C/L238S). Additional embodiments include mutations of the E. coli GBP at Y10C, N15C, Q26C, E93C, H152C, M182C, W183C, L255C, D257C, P294C, and V296C.

The GBPs of the presently disclosed subject matter may have an incorporated or attached marker(s) or reporter group(s). These covalently coupled reporter groups can couple glucose-mediated conformational transitions to changes in fluorescence or luminescent emission intensity, for example. In some embodiments, the GBP is labeled with a fluorescent or luminescent probe sensitive to protein environmental changes. Therefore, the amount of glucose in a sample may be measured by using a GBP with at least one attached fluorescent or luminescent probe. For example, a cysteine may be substituted for a wild-type amino acid in a GBP and the cysteine can be labeled with a fluorescent marker, such as acrylodan. As another example, rhodamine derivative tags can be covalently bound to cysteines, which are introduced at opposite ends of the glucose-binding cleft. When the GBP with the tags binds to glucose, the conformational change of the protein brings the two tags close enough so that they form dimers having altered excitation and fluorescence. As other examples, two halves of a luciferase or an aequorin protein may be fused to the two domains of the GBP. When the chimeric protein binds glucose, the conformational change results in the reassembly of the luminescent protein with the ability to emit bioluminescense.

Examples of fluorophores include, but are not limited to fluorescein, coumarins, rhodamines, 5-TMRIA (tetramethylrhodamine-5-iodoacetamide), o-aminobenzoic acid (ABZ), dinitrophenyl (DNP), 4-[(4-dimethylamino)phenyl]-azo)benzoic acid (DANSYL), 5- or 5(6)-carboxyfluorescein (FAM), 5- or 5(6)carboxytetramethylrhodamine (TMR), 5-(2-aminoethylamino)-1-naphthalenesulfonic acid (EDANS), 4-(dimethylamino)azobenzene-4′-carboxylic acid (DABCYL), 4-(dimethylamino)azobenzene-4′-sulfonyl chloride (DABSYL), nitro-Tyrosine (Tyr(NO₂)), Quantum Red®, Texas Red®, Cy3®, 7-nitro-4-benzofurazanyl (NBD), N-((2-iodoacetoxy)ethyl)-N-methyl)am-ino-7-nitrobenzoxadiazole (IANBD), 6-acryloyl-2-dimethylaminonaphthalene (acrylodan), pyrene, Lucifer Yellow, Cy5®, Dapoxyl® (2-bromoacetamidoethyl)sulfonamide, (N-(4,4-difluoro-1,3,5,7-tetramethyl-4-bora-3a,4a-diaza-s-indacene-2-yl)i-odoacetamide (Bodipy® 507/545 IA), N-(4,4-difluoro-5,7-diphenyl-4-bora-3a,4a-diaza-s-indacene-3-propionyl)-N-′-iodoacetylethylenediamine (BODIPY® 530/550 IA), 5-((((2-iodoacetyl)amino)ethyl)amino)naphthalene-1-sulfonic acid (1,5-IAEDANS), carboxy-X-rhodamine, 5/6-iodoacetamide (XRIA 5,6), eosin, acridine orange, Alexa Fluor 350®, Alexa Fluor 405®, Alexa Fluor 430®, Alexa Fluor 488®, Alexa Fluor 500®, Alexa Fluor 514®, Alexa Fluor 532®, Alexa Fluor 546®, Alexa Fluor 555®, Alexa Fluor 568®, Alexa Fluor 594®, Alexa Fluor 610®, Alexa Fluor 633®, Alexa Fluor 635®, Alexa Fluor 647®, Alexa Fluor 660®, Alexa Fluor 680®, Alexa Fluor 700® and Alexa Fluor 750®. Other luminescent labeling moieties include lanthanides, such as europium (Eu3+) and terbium (Tb3+), as well as metal-ligand complexes of ruthenium [Ru(II)], rhenium [Re(I)], or osmium [Os(II)], typically in complexes with diimine ligands, such as phenanthroline.

The fluorescent label can be attached to the mutated protein, for example an E. coli GBP, by any conventional means known in the art. For example, the reporter group may be attached via amines or carboxyl residues on the protein. Exemplary embodiments include covalent coupling via thiol groups on cysteine residues of the mutated or native protein. For example, for the mutated E. coli GBP, cysteines may be located at position 10, at position 11, position 14, at position 15, position 19, at position 26, at position 43, at position 74, at position 92, at position 93, position 107, position 110, position 112, at position 113, at position 137, at position 149, at position 152, at position 154, at position 182, at position 183, at position 186, at position 211, at position 213, at position 216, at position 238, at position 240, at position 242, at position 255, at position 257, at position 287, at position 292, at position 294, and at position 296. Representative mutations of binding proteins are described in U.S. Pat. No. 7,064,103 to Pitner et al., issued Jun. 20, 2006, U.S. Pat. No. 6,855,556 to Amiss et al., issued Feb. 15, 2005, and U.S. Patent Application Publication No. 2006/0280652, filed May 18, 2005, each of which is incorporated by reference in its entirety.

Any thiol-reactive group known in the art may be used for attaching reporter groups, such as fluorophores, to the cysteine in a natural or an engineered or mutated protein. For example, iodoacetamide, bromoacetamide, or maleimide are well known thiol-reactive moieties that may be used for this purpose.

A detected color change due to either a conformational change in the binding member, e.g., a binding protein, subsequent changes in the microenvironment of the dye, or both, can be correlated to the presence and/or amount, i.e., analyte concentration, of one or more target ligands or analytes. In some embodiments of the presently disclosed method, the binding protein undergoes a conformation change as a result of changes in ligand or analyte concentration of the sample suspected of containing one or more ligands or analytes. Accordingly, the method detects a color change as a result of changes in the ligand or analyte concentration. Such colors changes can provide a visually distinguishable color gradient over various glucose concentrations. The presently disclosed methods, for example, can exhibit a dynamic range of about 5 mg/dL to about 100 mg/dL, although methods exhibiting a broader or a narrower dynamic range are within the scope of the presently disclosed subject matter. In some embodiments, depending on the dye, the observed color of the dye changes from magenta (e.g., an absorption wavelength of about 550 nm) to blue (e.g., an absorption wavelength of about 590 nm) in the presence of D-glucose. In other embodiments, the observed color of the dye changes from orange (e.g., an absorption wavelength of about 490 nm) to red (e.g., an absorption wavelength of about 520 nm) in the presence of D-glucose. The color change can be observed with the naked eye to indicate the presence of, e.g., to provide a qualitative determination of, a ligand or analyte of interest in a sample. Further, in some embodiments, the color change can be correlated with the concentration of the ligand or analyte in the sample using simple instrumentation, for example, an absorption detection device, such as a photometer, or in some embodiments, a color wheel, to provide a quantitative determination of the ligand or analyte, e.g., glucose, in a sample. A representative glucose sensor is described in U.S. Patent Application Publication No. 2009/0104714, for Visual Glucose Sensor and Methods of Use Thereof, to J. Thomas, et al., published Apr. 23, 2009, which is incorporated herein by reference in its entirety.

The amount of one or more ligands or analytes present in a sample under test can be represented as a concentration. As used herein, the term “concentration” has its ordinary meaning in the art. The concentration can be expressed as a qualitative value, such as negative- or positive-type results, for example, as a “YES” or “NO” response indicating the presence or absence of a target analyte, or as a quantitative value, for example in units of mg/dL. Further, the concentration of a given analyte can be reported as a relative quantity or an absolute quantity. As used herein, “quantitative results” refer to results that provide absolute or relative values. The quantity (concentration) of a ligand or analyte can be equal to zero, indicating the absence of a particular ligand or analyte sought or that the concentration of the particular ligand or analyte is below the detection limits of the biosensor. The quantity measured can be the measured signal, e.g., a color change, without any additional measurements or manipulations. Alternatively, the quantity measured can be expressed as a difference, percentage or ratio of the measured value of the particular analyte to a measured value of another compound including, but not limited to, a standard or another ligand or analyte. The difference can be negative, indicating a decrease in the amount of measured ligand(s) or analyte(s). The quantities also can be expressed as a difference or ratio of the ligand(s) or analyte(s) to itself, measured at a different point in time. The quantities of ligands or analytes can be determined directly from a generated signal, or the generated signal can be used in an algorithm, with the algorithm designed to correlate the value of the generated signals to the quantity of ligands(s) or analyte(s) in the sample. The detection of the color change can be carried out continuously or intermittently at predetermined times allowing episodic or continuous sensing of an analyte, for example, glucose, to be performed.

The solution comprising the GBP that is to be dried in the methods of the presently disclosed subject matter can be a basic buffer, such as phosphate buffered saline (PBS), 3-(N-morpholino)propanesulfonic acid (MOPS), an ammonium carbonate buffer, and the like. It is believed that calcium depletion has an effect on the structure and thermal stability of the E. coli GBP, which can be restored by the binding of glucose to the GBP (D'Auria et al., 2005). Therefore, optionally, calcium is added to the buffer.

It was surprisingly found that glycerol is not required for the stability of the GBP during storage. It is well known in the art that high concentrations of glycerol in a storage buffer, such as 50% (v/v) glycerol, result in long-term stability of a protein stored at −20° C. or −70° C. The methods of the presently disclosed subject matter do not require glycerol to be added to either the solution comprised of the GBP before the GBP is dried or afterwards when the GBP is reconstituted.

The solution comprising the GBP also may or may not comprise a carbohydrate excipient. It is shown herein below that GBP without a carbohydrate excipient is still stable when dry formulations of the GBP are prepared. It was found that GBP lyophilized from a basic ammonium carbonate buffer without any additional components retained greater than 90% of its activity when stored in a sealed vial at −20° C. for a year (data not shown). An optional step in the methods includes adding at least one kind of carbohydrate excipient for further stability and/or activity of the GBP. In one embodiment, the carbohydrate excipient is a monosaccharide, disaccharide, trisaccharide, oligosaccharide, sugar alcohol, and the like. Examples of carbohydrate excipients that may be used in the methods include, but are not limited to, mannitol, sucrose, mannose, maltose, trehalose, raffinose, melezitose, lactitol, maltitol, starch, and the like. It was found that the addition of a carbohydrate excipient enhanced stability of the GBP during long-term storage.

The solution comprising the GBP also may comprise a substance that can form a matrix to stabilize the GBP. In some embodiments, the matrix is a hydrogel, which is a porous polymeric matrix that is filled with water. In some embodiments, the GBP solution comprising the hydrogel is polymerized, resulting in a covalent attachment of the hydrogel to the GBP, and then it is dried. In some embodiments, the polymerized formulation is stored for a period of time before the GBP is reconstituted and used to detect levels of glucose. The polymerized hydrogel may be stored for at least a day, at least a week, or at least a month. Without wishing to be bound to any one particular theory, it is believed that the glucose diffuses into the matrix to contact the GBP that is covalently bound to the matrix.

The solution comprising the GBP may be dried on a wide variety of materials, such as cellulose paper, glass fiber paper, polyethylene sheets, nitrocellulose paper, glass, plastic, and the like. The solution may be dried in any kind of container or on any surface, such as on a strip, in a plate, in a dish, in a well, in a microwell, and the like.

The methods of the presently disclosed subject matter comprise drying a solution comprising at least one GBP, wherein the GBP is active when reconstituted without any other reagents. In one embodiment, the GBP is dried by lyophilization or freeze drying. Lyophilization is a well known process in the art and it is used to store proteins for up to long periods of time. The steps of lyophilization involve an initial freezing step, where most of the solvent (typically water) is separated from the solutes. By the end of the freezing phase, the solute phase becomes highly concentrated. The second phase of lyophilization is the primary drying or ice sublimation phase, when the chamber pressure is reduced and the shelf temperature is raised to supply the heat removed by ice sublimation. In the third phase, the secondary drying phase, the water is desorbed from the solute phase, usually at an elevated temperature and low pressure.

The methods may include flash freezing or quick freezing the solution, for example in liquid nitrogen, before the lyophilization step. This extra step has the advantage of quickly freezing the sample so that there are less stresses imposed on the protein in the solution, especially proteins that are easily degraded or decomposed during the lyophilization process. In spray freeze drying, the solution is sprayed into a cold substance, such as liquid nitrogen, and the solution droplets are then lyophilized.

Other ways can be used to dry the formulation of GBP. For example, the GBP can be dried by vacuum drying. During this process, vacuum and heat are used to remove water or other solvents from a product to form a dry film. Other methods of drying the formulation may include spray drying, atmospheric freeze drying, carbon dioxide assisted nebulization with bubble drying (CAN-BD), lyophilization and other known to those skilled in the art.

The presently disclosed method for preparing a dry formulation of a GBP comprises drying a solution comprising at least one GBP, wherein the GBP is folded in an active conformation. After drying the solution of GBP, the GBP may be stored without a significant loss of activity. The loss of activity will depend on the temperature of storage of the dry formulation, the amount of time the dry formulation is stored, the liquid used to reconstitute the dry formulation, and the like.

Storage of the dried formulation of GBP can be at freezing temperatures, such as −70° C. or −20° C., at cold temperatures, such as in a refrigerator, at room temperature, or in warm temperatures, such as temperatures up to 50° C. In some embodiments, the dry formulation of GBP is stored at a temperature having a range from about 30° C. to about 50° C. Storage may be for as little time as minutes or it may be for longer than an hour, a day, a week, a month, a year, and any time in between. Storage may be in any kind of container, such as in a tube, flask, microwell, and the like. The choice of the storage container will depend on the size of the sample and the temperature at which storage will occur, among other factors.

The presently disclosed subject matter also provides dry formulations of GBP that are made by the process comprising drying a solution comprising at least one GBP and wherein the GBP is folded in an active conformation. The presently disclosed compositions can be assembled into kits for use in measuring glucose levels of a sample.

B. Methods of Determining Glucose Concentrations Using GBP

The presently disclosed subject matter provides methods for determining the amount or concentration of glucose in a sample. The methods comprise drying a solution comprising at least one GBP, wherein the GBP is folded in an active conformation, reconstituting the GBP with a liquid sample, and measuring the amount of glucose in the liquid sample.

The liquids that can be used to reconstitute the dry formulation do not require extra components, such as glycerol, carbohydrate excipients, and the like. The dry formulation of GBP may be directly reconstituted into a basic buffer or into a biological sample, such as plasma, diluted blood, interstitial fluid, urine and the like. The choice of liquid will depend in part on the subsequent use of the GBP. In some embodiments, the reconstituted GBP is used to measure the amount of glucose directly in the liquid that is used for reconstitution of the GBP. In other embodiments, the GBP will be reconstituted in one liquid and then transferred into another liquid for measuring the glucose levels. Under optimal conditions, the rehydration occurs quickly so as to minimize any deleterious structural changes to the GBP.

In some embodiments, the sample can be pre-treated prior to use, such as preparing plasma from blood, diluting viscous fluids, or the like. Such methods of treatment can involve dilution, filtration, distillation, concentration, inactivation of interfering compounds, and the addition of reagents.

The sample can be any sample obtained from a subject. The term “subject” refers to an organism, tissue, or cell from which a sample can be obtained. A subject can include a human subject for medical purposes, such as diagnosis and/or treatment of a condition or disease, or an animal subject for medical, veterinary purposes, or developmental purposes. A subject also can include sample material from tissue culture, cell culture, organ replication, stem cell production and the like. Suitable animal subjects include mammals and avians. The term “avian” as used herein includes, but is not limited to, chickens, ducks, geese, quail, turkeys, and pheasants. The term “mammal” as used herein includes, but is not limited to, primates, e.g, humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. Preferably, the subject is a mammal or a mammalian cell. More preferably, the subject is a human or a human cell. Human subjects include, but are not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. A subject also can refer to cells or collections of cells in laboratory or bioprocessing culture in tests for viability, differentiation, marker production, expression, and the like.

As disclosed hereinabove, covalently coupled reporter groups to GBP can couple glucose-mediated conformational transitions to changes in fluorescence emission intensity. This kind of modified GBP can be dried using the methods of the presently disclosed subject matter, reconstituted, and used as a glucose biosensor. As used herein, the term “biosensor” generally refers to a device that undergoes a detectable change in specific response to the presence of a ligand or analyte. Such biosensors combine the molecular recognition properties of biological macromolecules, such as a binding protein, with environmentally-sensitive dyes that produce a detectable color change upon ligand or analyte binding. The detectable color change can include a shift in the absorption wavelength, a change in intensity, or a combination thereof. Accordingly, a biosensor translates a binding event into a directly measurable photometric or colorimetric property.

At least one advantage of using a GBP in a glucose biosensor is that no additional reagents are needed for the GBP to measure or sense the amount of glucose in the environment. Glucose oxidase sensors have the disadvantage of oxygen potentially becoming a limiting reagent. Another disadvantage of glucose oxidase sensors is that they produce hydrogen peroxide in the presence of glucose that in time degrades the enzyme itself. In contrast, a GBP sensor does not alter the chemistry of glucose. Instead, a GBP sensor measures the equilibrium between association and dissociation of the GBP and glucose molecules in its environment. Therefore, a GBP sensor can be continuous, such that it monitors glucose in its environment at multiple time points, or it can be a single time point sensor, such that only one measurement is taken.

In some embodiments, a GBP biosensor can measure micromolar concentrations of glucose. This high sensitivity to glucose allows the measurement of low glucose concentrations, for example in the micromolar range, such as in extracted interstitial fluid.

### II. DEFINITIONS

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The descriptions and specific examples that follow are only intended for the purposes of illustration, and are not to be construed as limiting in any manner to make compounds of the disclosure by other methods.

### Example 1

### Preparation of Stable, Dry GBP Formulations by Freeze Process Techniques

In several of the Examples shown herein, an engineered dye labeled GBP 3M-NBD was used. A fluorescent-labeled triple mutant of the E. coli GBP (“the 3M protein”) was prepared as follows. The 3M protein is an E. coli GBP protein (GenBank Accession No. P02927, without the 23 amino acid leader sequence), and where a cysteine is substituted for an glutamic acid at position 149, an arginine is substituted for an alanine at position 213, and a serine is substituted for leucine at position 238 (E149CA213RL238S). The 3M protein was labeled with IANBD, and the NBD-labeled 3M protein was prepared as described in U.S. application Ser. No. 10/040,077, filed Jan. 4, 2002, now U.S. Pat. No. 6,855,556, and Ser. No. 11/077,028, filed Mar. 11, 2005, and published as United States Pre-grant Publication 2005/0239155, both of which are incorporated herein by reference in their entirety.

In this Example, a solution of GBP 3M-NBD (10 μM), Texas Red-BSA (2.5 μM), MOPS (10 mM), CaCl₂ (1 mM), NaCl (10 mM), and trehalose (40 mg/mL), pH 7.4 was prepared. Samples from this solution were taken to process by lyophilization with a typical cooling ramp of −40° C. on a shelf lyophilizer (Lyo), by a Quench Freeze in liquid nitrogen followed by lyophilization (QF Lyo), or by undergoing the BDT Aerodynamically Light Particle (ALP) process (spray freeze drying; SFD). With the SFD process, 7.2 mL GBP solution was sprayed from an AccuSpray nozzle into 100 mL liquid N₂ and dried by lyophilization. The lyophilized samples were reconstituted using PBS buffer. The original GBP solution (Solution) and excipient-free lyophilized GBP (Protein; prepared by Paragon Bioservices, Baltimore, Md.), were tested along with the other samples. Freshly prepared protein controls exhibited a QF of 7.49±0.60 for the duration of the study.

Stability was evaluated at 0, 2, 4, 8, and 12 weeks at both room temperature (mean of 21.8; SDEV of 0.4° C.) and 34° C. (mean of 34.5, SDEV of 1.3° C.). GBP activity was evaluated by measuring solution phase protein activity (or dynamic signal response), QF (background subtracted fluorescence in presence of 90 mM glucose)/background subtracted fluorescence in presence of 0 mM glucose). Since a Q_{F} of 1 equates to no residual activity, the % Activity Loss was calculated as (Q_{FT2}−Q_{FT1})/(Q_{FT1}−1)×100, where Q_{FT2} and Q_{FT1} correspond to activities at the second time point (T₂), and the first time point (T₁) respectively. Therefore, remaining % activity=100−% Activity Loss.

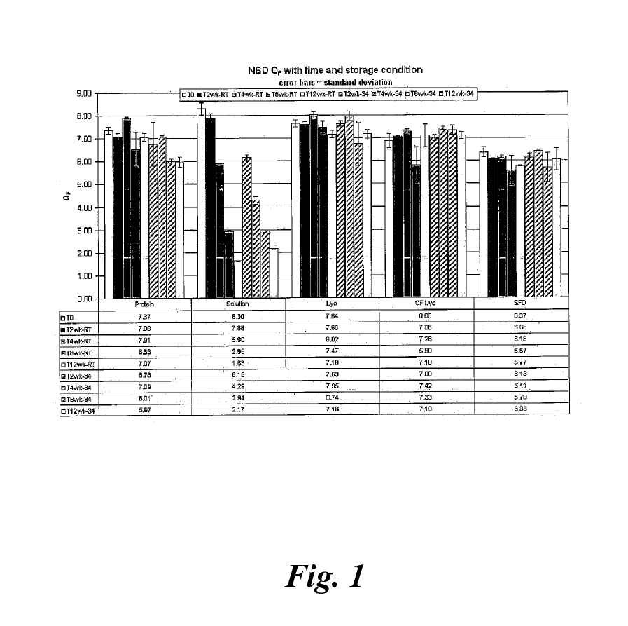

FIG. 1 shows that after 12 weeks, GBP in solution (Solution) had a mean % Activity Loss of 92% at room temperature (T12wk-RT) and 84% at 34° C. (T12wk-34). Most of the losses occurred in the first 4 weeks. In contrast, mean Activity Losses during storage for the dry formulations were all below 12% after 12 weeks at either room temperature (Lyo, QF Lyo, SFD, lanes T12wk-RT) or at 34° C. (Lyo, QF Lyo, SFD, lanes T12wk-34). In contrast to the Protein sample, the Lyophilized and Quench Frozen followed by Lyophilization samples also showed an improvement in retained activity for storage at 34° C. (Protein compared to Lyo and QF Lyo). Activity losses due to processing were −9% for the Lyophilized samples, −19% for the Quench Frozen followed by Lyophilization samples, and −26% for the SFD samples.

These data show that excipient-free lyophilized GBP (Protein) can be dried and stored without loss of most of its activity. Addition of a carbohydrate excipient to the buffer prior to drying the solution, such as trehalose as shown in this experiment, increased the amount of GBP activity after storage as compared to the Protein sample without the addition of trehalose.

### Example 2

### Preparation of Stable, Dry GBP Hydrogel Formulations Suitable for Direct Sensing

To prepare dry GBP-hydrogel formulations suitable for direct sensing after 12 weeks, polyethylene glycol dimethacrylate (200 μL), a 6-Arm PEG (each arm terminated with an acrylate ester, BioLink, NC, MW 10,000, 2-2.5 mg), 2-hydroxy-2-methylpropiophenone (HMPP, 1-2 μL), MOPS buffer (3-morpholinopropanesulfonate, final concentration of 13.3 mM), CaCl₂ (final concentration 0-1.3 mM), NaCl (final concentration 13-185 mM), GBP W183C Acrylodan (a GBP with tryptophan 183 substituted with a cysteine attached to an acrylodan fluorescent marker; final concentration 270 μM), Trehalose (64-320 mg), and Triton X-45 (0-2.5 μL) were combined and mixed to give 4 mL of a homogeneous solution. The solution was applied to a silanized glass plate, 250-μm spacers placed, and a second glass plate added to cover the prepolymer solution so that a liquid film formed between the plates. The prepolymer film was then polymerized under a 500 W Tungston lamp (300-nm cutoff filter) and 4-mm disks were excised. The disks were dried using a lyophilizer for 16 h and stored in a low water vapor transmission rate (WVTR) package with dessicant. A portion of the disks were placed in microwell plates and assayed in PBS containing various concentrations of glucose. These results were used to obtain initial calibration parameters (K_{D}, Ro, and Q_{R}*); fluorescence was measured: Ex=380-410 nm, Em at 450-490 nm (F_{b}) and 510-550 nm (F_{g}); Rₒ is the fluorescence ratio (F_{g}/F_{b}) at 0 mM glucose; QR* is the ratio of (R_{∞}/Rₒ) where R∞ is the theoretical fluorescence ratio (F_{g}/F_{b}) at infinite glucose; QR* may be estimated from fitting the binding curve. The remaining disks were then stored at room temperature for 12 weeks and tested as before using the original calibration parameters to convert fluorescence ratios to glucose concentration. Mean Performance Errors ranged from 5-20% using 5 replicate measurements at 2.5 (45), 5 (90), 10 (180), 20 (360), 30 (540) mM (mg/dL) glucose.

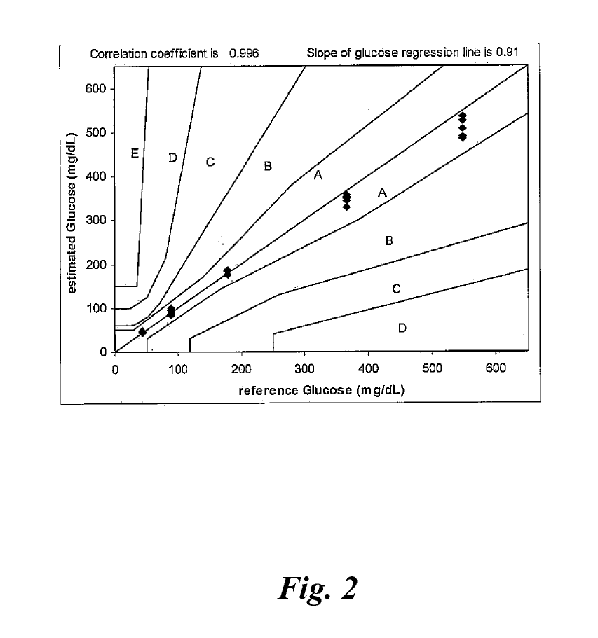

Representative data are presented in FIG. 2 and show that after 12 weeks of storage, the dry GBP hydrogel formulations were capable of sensing the different concentrations of glucose added to the assay (A: Self-monitoring of blood glucose (SMBG)<20% deviation from true blood glucose (BG) or both SMBG and BG<70 mg/dl); B: deviation from true BG>20% but leads to no treatment or benign treatment; C: overcorrection of acceptable BG levels; D: dangerous failure to detect and treat BG errors, and E: erroneous treatment; Parkins et al., 2000).

### Example 3

### Preparation and Performance of Stable, Dry GBP Film Formulations in Microwell Plates

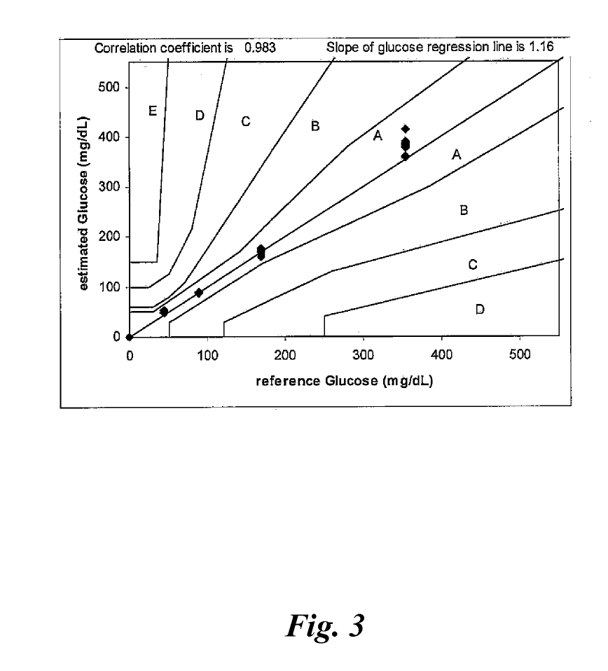

To prepare dried GBP formulations in microwell plates, Glucose Binding Protein 3M-NBD was dissolved to a final concentration of 62.5 μM in PBS containing 3.25 mM CaCl₂ and Trehalose (2.5 wt %); Texas Red-Bovine Serum Albumin (TR-BSA, 7.6 μM BSA to obtain 4:1 NBD:TR) was included as an internal reference. A 40 μL volume of this solution was dispensed into each well of a 96 microwell plate(s) and dried under stepped vacuum with a shelf temperature of 35° C. for a total drying cycle of 21 hrs. Dry plates could be calibrated with standard glucose solutions (rehydration volume=100 μL), and maintained calibrated stability for 6 months when stored dessicated in low WVTR pouches at room temperature. After 24 weeks, retest of known glucose solutions using the initial calibration constants and normalized Rₒ gave a 9% Mean Performance Error (MPE) (FIG. 3). These data show that the GBP hydrogel formulations can be prepared in microwell plates, stored for an extended period of time, and are still capable of sensing the different concentrations of glucose.

### Example 4

### Use of Stable, Dry GBP Film Formulations for a Rapid Glucose Assay in Human Plasma

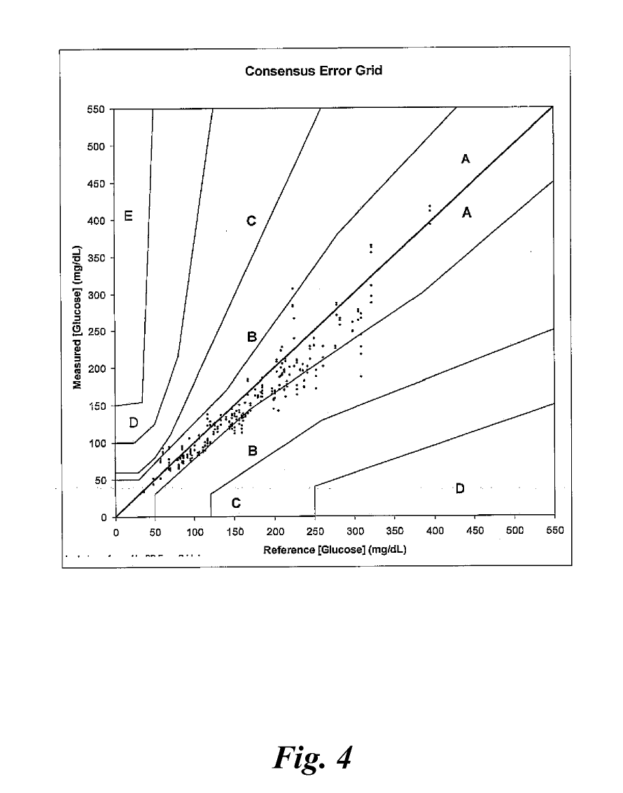

This Example describes the use of stable, dry GBP formulations for a rapid glucose assay in 100% human plasma without reagent additions. Microwell plates were prepared as described in Example 3 except that Glucose Binding Protein W183 Acrylodan (100 μM) was substituted for GBP 3M-NBD and no TR-BSA was used in this formulation. After drying, 50 μL aliquots of serum were dispensed into the microwells and the plate fluorescence was evaluated on a BMG fluorometer at 10-20 minutes post addition. GBP calibrations were developed using 10 randomly selected human plasma specimens from human subjects afflicted with diabetes (these calibration specimens had glucose values distributed throughout the measurement range). When an additional 100 human plasma specimens obtained from human subjects afflicted with diabetes were evaluated using this method, the Mean Performance Error (MPE) was 13% versus reference values obtained from a Yellow Springs Instrument (YSI) clinical glucose analyzer. Analytical performance of the dried microwell formulation was comparable to that obtained at the time of collection using a BD Logic glucose meter (13% MPE). There were no outliers for either the BD Logic glucose meter assay or the dried formulation microwell assay versus YSI, and all dry GBP formulation results were in the clinically acceptable A+B range of a Consensus Error Grid. Dry GBP formulation assays were performed in triplicate and are plotted in FIG. 4. Similar performance was obtained using GBP 3M-NBP with TR-BSA added as described in Example 3.

As seen in FIG. 4, GBP film formulations can be used to detect accurate levels of glucose in human plasma samples without the addition of any reagents.

### Example 5

### Preparation of Polyethylene Glycol-GBP Hydrogel Disks using Sorbitol as a Stabilizing Agent

This Example describes the preparation of PEG-GBP disks using sorbitol as a stabilizing agent. In a glass container, 200 μL of Polyethylene Glycol 1000 Dimethacrylate PEGDMA 1000 (Degussa Product #6874-0), 2.5 μL of 6-Arm PEG with acrylate terminations (Biolink Product # BLS-018-083), 1 μL of 2-Hydroxy-2-methylpropiophenone (Photo Initiator PI) (Aldrich Product #405655), 0.0165 g of sorbitol (Aldrich Product # S1876) and 450 μL of Phosphate Buffer (PBS) were vortexed for about 30 seconds, or until the reagents were well mixed. 150 μL of GBP 3M-NED Solution (2 mg/mL) were added and the mixture was vortexed for 5 seconds. Two microscope slides with 1-mm spacers at the ends were clamped with a small binder clip. The pre-polymer solution was poured in between the plates and polymerized under UV light (500 Watts, 300 nm high pass filter) for 3.5 minutes. The slides were separated and disks punched with a #6 punch. Disks were selected for the initial assay and one disk was added per well in the well plate. 100 μL of PBS containing various concentrations of glucose was added to each well. After fifteen minutes the plates were read in a Cytofluor microwell fluorometer (Ex 485 nm, Em 535 nm). The rest of the polymerized film was covered with the cover slide and the ends taped together without applying too much pressure. The polymerized film/slide sandwich was cooled at −70° C. for at least one hour, and then lyophilized overnight. The disks were punched from the lyophilized film with a #4 punch, reconstituted in PBS buffer, and tested as described above,

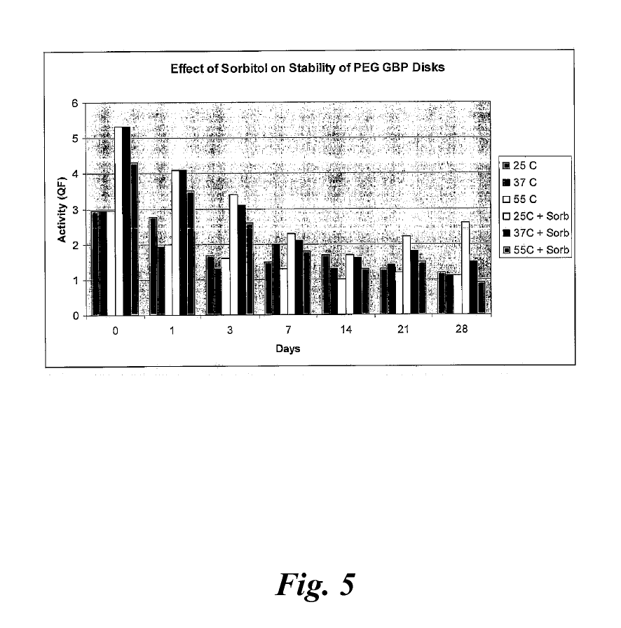

FIG. 5 shows that the addition of sorbitol to the GBP solution resulted in increased activity of GBP from initial incubation to 28 days of storage. This trend was generally consistent across all of the temperatures tested. Therefore, the addition of sorbitol improved the stability of the PEG-GBP formulations.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.
D'Auria, S., Ausili, A., Marabotti, A., Varriale, A., Scognamiglio, V., Staiano, M., Bertoli, E., Rossi, M., and Tanfani, F., Binding of glucose to the D-galactose/D-glucose-binding protein from Escherichia coli restores the native protein secondary structure and thermostability that are lost upon calcium depletion. J. Biochem. 2006, 139(2):213.
Lai, M. C. and Topp, E. M., Solid-state chemical stability of proteins and peptides. J. Pharm. Sci. 1999, 85(5) 489.
Parkins, J. L., Slatin, S. L., Pardo, S., and Ginsberg, B. H., A new consensus error grid to evaluate the clinical significance of inaccuracies in the measurement of blood glucose. Diabetes Care. 2000, 23(8) 1143.
Richards, A. B., Krakowka, S., Dexter, L. B., Schmid, H., Wolterbeek, A. P., Waalkens-Berendsen, D. H., Shigoyuki, A., and Kurimoto, M., Trehalose: a review of properties, history of use and human tolerance, and results of multiple safety studies. Food Chem Toxicol. 2002, 40(7) 871.
Stepanenko, O. V., Fonin A. V., Stepanenko, O. V., Morozova, K. S., Verkhusha, V. V., Kuznetsova, I. M., Turoverov, K. K., Staiano, M., D'Auria, S., New insight in protein-ligand interactions. 2. Stability and properties of two mutant forms of the D-galactose/D-glucose-binding protein from E. coli. J. Phys Chem B., 2011, 115(29) 9022.
U.S. Pat. No. 4,891,319 to Roser, for Protection of Proteins and the Like; and
U.S. Pat. No. 7,956,160 to Krishnan et al., for Concentrated Protein Lyophilates, Methods, and Uses.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A method for preparing a dry formulation of a Glucose Binding Protein (GBP), the method comprising:
drying a solution comprising at least one GBP, and
wherein the GBP is folded in an active conformation.

2. The method of claim 1, wherein the GBP does not lose more than 20% activity as compared to the GBP in the original solution.

3. The method of claim 1, wherein the solution does not comprise glycerol.

4. The method of claim 1, wherein lyophilization is used to dry the solution.

5. The method of claim 4, wherein the solution is frozen in liquid nitrogen before lyophilization.

6. The method of claim 5, wherein the solution is sprayed into liquid nitrogen before lyophilization.

7. The method of claim 1, wherein vacuum drying is used to dry the solution.

8. The method of claim 1, wherein the solution further comprises at least one carbohydrate excipient.

9. The method of claim 8, wherein the at least one carbohydrate excipient is selected from the group consisting of a monosaccharide, a disaccharide, a trisaccharide, an oligosaccharide, and a sugar alcohol.

10. The method of claim 9, wherein the at least one kind of carbohydrate excipient is selected from the group consisting of trehalose and sorbitol.

11. The method of claim 1, wherein the dry formulation of GBP is stored.

12. The method of claim 11, wherein the dry formulation of GBP is stored at room temperature.

13. The method of claim 11, wherein the dry formulation of GBP is stored at a temperature having a range from about 30° C. to about 50° C.

14. The method of claim 11, wherein the dry formulation of GBP is stored for more than one day.

15. The method of claim 1, wherein the dry formulation of GBP is reconstituted in a liquid.

16. The method of claim 15, wherein the liquid comprises a biological sample.

17. The method of claim 15, wherein the reconstituted GBP is used to determine the glucose concentration of a sample.

18. The method of claim 1, wherein the solution is dried in a microwell.

19. The method of claim 1, wherein the solution further comprises a hydrogel formulation.

20. The method of claim 19, wherein the solution comprising a hydrogel formulation is polymerized and then dried.

21. The method of claim 20, wherein the polymerized hydrogel is stored for at least a day before the GBP is reconstituted.

22. A dry formulation of GBP made by the process comprising:
drying a solution comprising at least one GBP; and
wherein the GBP is folded in an active conformation.

23. A kit comprising the dry formulation of claim 22.

24. A method for determining an amount of glucose in a liquid sample, the method comprising:
drying a solution comprising at least one GBP, wherein the GBP is folded in an active conformation;
reconstituting the GBP with a liquid sample; and
measuring the amount of glucose in the liquid sample.

25. The method of claim 24, wherein the amount of glucose is measured using a GBP having at least one fluorescent probe attached thereto.

26. The method of claim 24, wherein the amount of glucose is measured using a GBP having at least one luminescent probe attached thereto.

27. The method of claim 24, wherein the solution does not comprise glycerol.

28. The method of claim 24, wherein lyophilization is used to dry the solution.

29. The method of claim 28, wherein the solution is frozen in liquid nitrogen before lyophilization.

30. The method of claim 29, wherein the solution is sprayed into liquid nitrogen before lyophilization.

31. The method of claim 24, wherein vacuum drying is used to dry the solution.

32. The method of claim 24, wherein the solution comprises at least one carbohydrate excipient.

33. The method of claim 32, wherein the at least one carbohydrate excipient is selected from the group consisting of a monosaccharide, a disaccharide, a trisaccharide, an oligosaccharide, and a sugar alcohol.

34. The method of claim 33, wherein the at least one carbohydrate excipient is selected from the group consisting of trehalose and sorbitol.

35. The method of claim 24, wherein the dry GBP is stored.

36. The method of claim 35, wherein the dry GBP is stored at room temperature.

37. The method of claim 35, wherein the dry GBP is stored for more than one day.

38. The method of claim 24, wherein the dry GBP is reconstituted in a biological sample.

39. The method of claim 24, wherein the solution is dried in a microwell.

40. The method of claim 24, wherein the solution comprises a hydrogel formulation.

41. The method of claim 40, wherein the solution comprising a hydrogel formulation is polymerized and then dried.

42. The method of claim 41, wherein the polymerized hydrogel is stored for at least one day before the GBP is reconstituted.
